(19) United States
(12) Patent Application Publication
(10) Pub. No.: US 2025/0070458 A1
(43) Pub. Date: Feb. 27, 2025

(54) Antenna Assembly for Glucose Monitoring Device

(71) Applicant: KYOCERA AVX Components (San Diego), Inc., San Diego, CA (US)

(72) Inventors: Weichun Eric Lin, San Diego, CA (US); Jin-ho Ahn, San Diego, CA (US)

(21) Appl. No.: 18/943,260

(22) Filed: Nov. 11, 2024

Related U.S. Application Data
(63) Continuation-in-part of application No. 18/517,887, filed on Nov. 22, 2023.
(60) Provisional application No. 63/427,619, filed on Nov. 23, 2022.

(51) Int. Cl. H01Q 1/27; A61B 5/145; H01Q 1/48; H01Q 9/04

(52) U.S. Cl. CPC H01Q 1/273; A61B 5/14532; H01Q 1/48; H01Q 9/0407

(57) ABSTRACT

A glucose monitoring device is provided. The glucose monitoring device includes a housing having a first portion and a second portion, the second portion operable to be placed adjacent to a human body during operation of the glucose monitoring device. The glucose monitoring device further includes a circuit board positioned between the first portion and the second portion of the housing, the circuit board having a trace antenna. The glucose monitoring device further includes a conductive structure spaced apart from the circuit board.

### PRIORITY CLAIM

The present application is a continuation in part (CIP) of commonly owned U.S. application Ser. No. 18/517,887, titled “Antenna Assembly for Glucose Monitoring Device,’ filed on Nov. 22, 2023, which claims the benefit of priority of U.S. Application Ser. No. 63/427,619, titled “Antenna Assembly for Glucose Monitoring Device,” filed Nov. 23, 2022, both of which are incorporated herein by reference.

### FIELD

The present disclosure relates generally to an antenna assembly, and more particularly to an antenna assembly for a glucose monitoring device.

### BACKGROUND

Antennas can be used to facilitate wireless communication between devices. It can be desirable for antennas to operate as with a high antenna radiation efficiency to improve wireless communication between devices. Antennas may need to be incorporated into a variety of different types of devices to provide for wireless communication. With the advance of medical device technology, antennas are being incorporated into a variety of different medical devices, such as glucose monitoring devices.

### SUMMARY

Aspects and advantages of embodiments of the present disclosure will be set forth in part in the following description, or may be learned from the description, or may be learned through practice of the embodiments.

One example aspect of the present disclosure is directed to a glucose monitoring device. The glucose monitoring device includes a housing having a first portion and a second portion, the second portion operable to be placed adjacent to a human body during operation of the glucose monitoring device. The glucose monitoring device further includes a circuit board positioned between the first portion and the second portion of the housing, the circuit board having a trace antenna. The glucose monitoring device further includes a conductive structure spaced apart from the circuit board.

Another example aspect of the present disclosure is directed to an antenna assembly for a glucose monitoring device. The antenna assembly includes a circuit board positioned between a first portion and a second portion of a housing, the second portion of the housing operable to be placed adjacent to a human body during operation of the glucose monitoring device. The antenna assembly further includes a trace antenna associated with the circuit board. The antenna assembly further includes a conductive structure positioned between the second portion of the housing and the circuit board. The conductive structure is spaced apart from the circuit board.

Another example aspect of the present disclosure is directed to a glucose monitoring device. The glucose monitoring device includes a plastic housing having a first portion and a second portion, the second portion operable to be placed adjacent to a human body during operation of the glucose monitoring device. The glucose monitoring device further includes a circuit board having a trace antenna positioned between the first portion and the second portion. The glucose monitoring device further includes a laser direct structuring (LDS) conductive structure formed on the second portion of the housing. The LDS conductive structure is spaced apart from the circuit board.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts an exploded view of an example antenna assembly for a glucose monitoring device according to example embodiments of the present disclosure;

FIG. 2 depicts a stamped metal antenna according to example embodiments of the present disclosure;

FIG. 3 depicts a cross-sectional view of an example glucose monitoring device according to example embodiments of the present disclosure;

FIG. 4 depicts example antenna radiation efficiency for an antenna assembly according to example embodiments of the present disclosure;

FIG. 5 depicts an exploded view of an example antenna assembly for a glucose monitoring device according to example embodiments of the present disclosure; and

FIGS. 6A-6G depict various conductive structures of an antenna assembly according to example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations. As used herein, the use of the term “about” in conjunction with a numerical value is intended to refer to within 10% of the numerical value.

Example aspects of the present disclosure are directed to an antenna assembly for use in, for instance, a glucose monitoring device. Integrating antennas into devices that are worn or placed near a human body can pose challenges due to degradation of RF signals by the human body. According to example embodiments of the present disclosure, the antenna assembly may include a laser direct structuring (LDS) or stamped metal conductive structure that is positioned between an antenna (e.g., trace antenna) of the glucose monitoring device and a human body during operation. Specifically, the LDS or stamped metal conductive structure is configured to decouple the human body from the antenna to lessen the body effect caused by the human body during operation of the device.

In some embodiments, the circuit board of the glucose monitoring device may have an antenna, such as a trace antenna. The LDS antenna or stamped metal conductive structure (e.g., antenna) may act as a ground plane for the trace antenna. For instance, the laser direct structuring (LDS) or stamped metal conductive structure may have planar shape that nearly or generally conforms to a shape of the circuit board. The laser direct structuring (LDS) or stamped metal conductive structure can have an opening configured to accommodate a structure of the glucose monitoring device, such as a sensor operable to be inserted into a human body during operation of the glucose monitoring device. The laser direct structuring (LDS) or stamped metal conductive structure may have a planar portion with a substantially continuous and/or uninterrupted metal.

In some embodiments, the conductive structure may have one or more tabs, such as five tabs. The tabs may be configured to operably couple the conductive structure to the circuit board, such that the conductive structure may act as a ground plane for the antenna (e.g., trace antenna) of the circuit board. Accordingly, the stamped or LDS conductive structure may have one or more tabs to secure the stamped metal antenna to the circuit board.

In alternative embodiments, the laser direct structuring (LDS) or stamped metal conductive structure may be spaced apart from and not connected to the circuit board of the glucose monitoring device. For instance, a circuit board may have a trace antenna and a ground plane associated with the trace antenna. The laser direct structuring (LDS) or stamped metal conductive structure may be spaced apart from the ground plane associated with the circuit board such that the laser direct structuring (LDS) or stamped metal conductive structure is not connected to the ground plane associated with the circuit board. For instance, the laser direct structuring (LDS) or stamped metal ground plane may be positioned on (e.g., formed on) a bottom portion of the housing such that the laser direct structuring (LDS) or stamped metal conductive structure is spaced apart from (e.g., not connected to) the ground plane associated with the circuit board of the glucose monitoring device.

The antenna assembly according to example embodiments of the present disclosure may provide technical effects and benefits. For instance, laser direct structuring (LDS) or stamped metal conductive structure that is positioned between an antenna (e.g., trace antenna) of the glucose monitoring device and a human body during operation may decouple the human body from the antenna to lessen the body effect caused by the human body during operation of the device. This may result in improved antenna radiation efficiency for the antenna assembly at frequencies in, for instance, the 2.4 GHz band. In addition, a

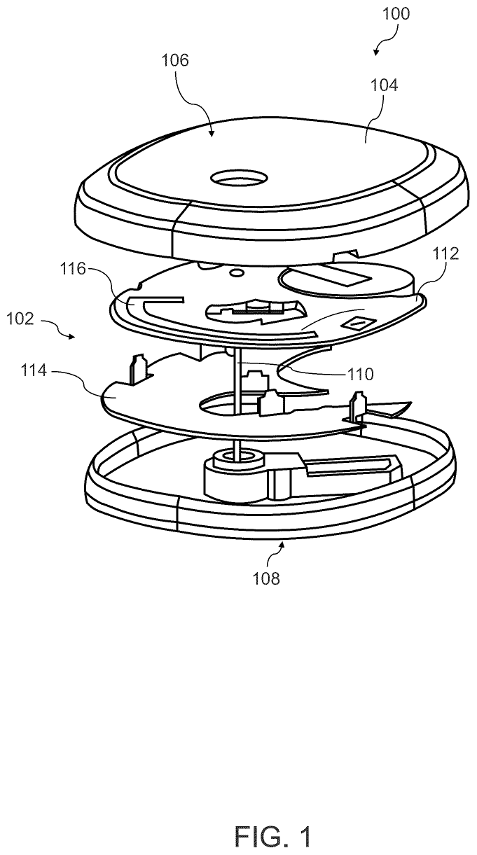

FIG. 1 depicts an exploded view of an example glucose monitoring device 100 including an antenna assembly 102 according to example embodiments of the present disclosure. The glucose monitoring device 100 may include a housing 104, such as a dielectric housing. The housing 104 may have a first surface 106 and a second surface 108 opposite the first surface 106. The second surface 108 may be operable to be placed adjacent to a human body during operation of the glucose monitoring device 100. For instance, an adhesive patch may be positioned between second surface 108 and a human body during operation of the glucose monitoring device 100, such that second surface 108 is held in place adjacent to the human body during operation of the glucose monitoring device 100.

The glucose monitoring device 100 may have a sensor 110. The sensor 110 may extend at least partially from the second surface 108 into or just below the skin of a human body in operation. The sensor 110 may provide for continuous glucose monitoring.

The glucose monitoring device 100 may include a circuit board 112 within the housing 104. The circuit board 112 may include various components/electronics associated with the glucose monitoring device 100.

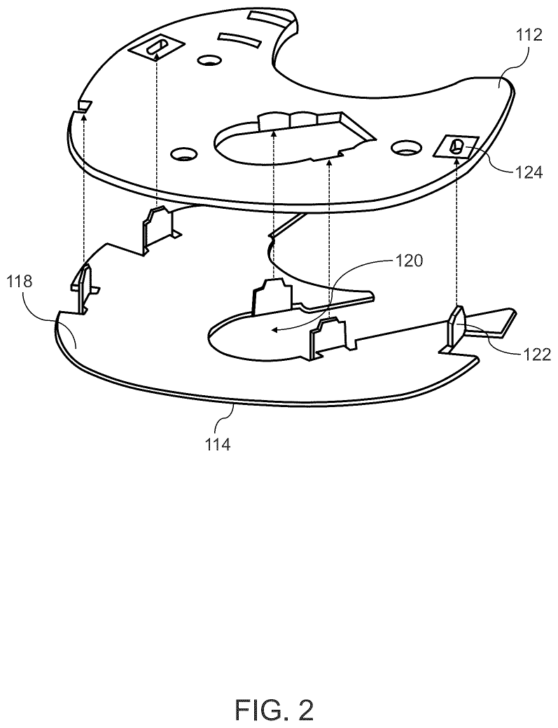

The antenna assembly 102 may include an antenna, such a stamped metal antenna 114. The stamped metal antenna 114 may be disposed between the circuit board 112 and the second surface 108. The stamped metal antenna 114 may include a planar portion (described with reference to FIG. 2) that acts as a ground plane for the antenna assembly 102.

The antenna assembly 102 may include an antenna 116 on the circuit board 112, such as a trace antenna. The stamped metal antenna 114 may include a planar portion (described with reference to FIG. 2) that acts as a ground plane for the antenna 116. While antenna 114 is generally described as a stamped metal antenna 114, those of ordinary skill in the art will understand that antenna 114 may be a different type of antenna without deviating from the scope of the present disclosure. For instance, antenna 114 may be a laser direct structuring (LDS) antenna 114 in some embodiments.

FIG. 2 depicts a close-up exploded view of the circuit board 112 and the stamped metal antenna 114. As illustrated, the stamped metal antenna 114 has a shape that generally conforms to a shape of the circuit board 112. The stamped metal antenna 114 may include a substantially continuous and/or uninterrupted metal planar portion 118. The metal planar portion 118 may be uninterrupted for at least about 90% of the total surface area of the stamped metal antenna 114, such as about 100% of the total surface area of the stamped metal antenna 114. The stamped metal antenna 114 may include an opening to accommodate the sensor 110 (FIG. 1) for the glucose monitoring device 100.

The stamped metal antenna 114 may have one or more tabs 122 to mechanically connect the stamped metal antenna 114 to the circuit board 112. The one or more tabs 122 may be received into openings 124 of the circuit board 112. The example stamped metal antenna 114 of FIG. 2 has five tabs for connection to the circuit board 112.

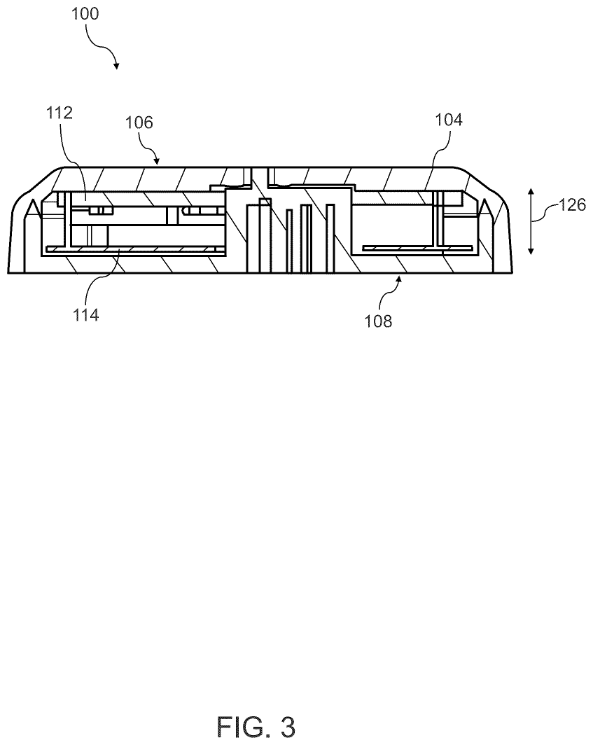

FIG. 3 depicts a cross-sectional view of an example glucose monitoring device 100 according to example embodiments of the present disclosure. As shown in FIG. 3, the stamped metal antenna 114 is separated from the circuit board 112 by a distance 126 when connected to the circuit board 112. In some embodiments, the distance 126 may be less than about 2.9 mm, such as less than about 2.5 mm, such as less than about 2.25 mm, such as in a range of about 1 mm to about 2.5 mm.

As shown in FIG. 3, the stamped metal antenna 114 is disposed in a spaced apart relationship from the circuit board 112 and from an antenna 116 (e.g., trace antenna) disposed on the circuit board 112 in a direction towards the second surface 108. The second surface 108 of the glucose monitoring device 100 is operable to be placed adjacent to a human body during operation of the glucose monitoring device 100.

The antenna assembly 102 (FIG. 1) may be operable to communicate signals in the 2.4 GHz frequency band. In some examples, the antenna assembly 102 may be operable to communicate signals with a remote device (e.g., a smartphone or other device) via a short-range wireless communication protocol (e.g., Bluetooth communication protocol). In some examples, the antenna assembly 102 may be operable to communicate signals with a remote device (e.g., a smartphone or other device) via an 802.11 communication protocol. Other suitable communication protocols may be used without deviating from the scope of the present disclosure.

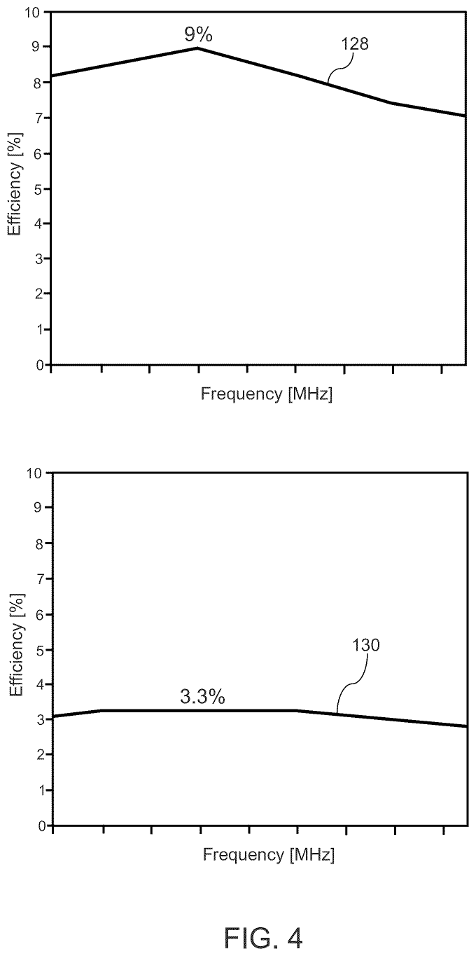

FIG. 4 depicts a plot of example antenna radiation efficiency for an antenna assembly for use with a glucose monitoring device according to example embodiments of the present disclosure. FIG. 4 plots frequencies in the 2.4 GHz frequency band along the horizontal axis. FIG. 4 plots antenna radiation efficiency along the vertical axis. Curve 128 is associated with the antenna assembly 102 wherein the stamped metal antenna 114 is disposed between the circuit board 112 and the second surface 108 of a glucose monitoring device 100 placed adjacent to a human body during operation of the glucose monitoring device 100. Curve 130 is associated with an antenna assembly that is placed between the circuit board 112 and an opposing first surface 106 of a glucose monitoring device that is opposite the surface 108 placed adjacent to the human body.

As illustrated in FIG. 4 by curve 128, the antenna assembly 102 may demonstrate an antenna radiation efficiency of at least about 6%, such as at least about 9% in the 2.4 GHZ frequency band. This provides improvement over the antenna represented by curve 130, which only demonstrates an antenna radiation efficiency of about 3.3% in the 2.4 GHz frequency band.

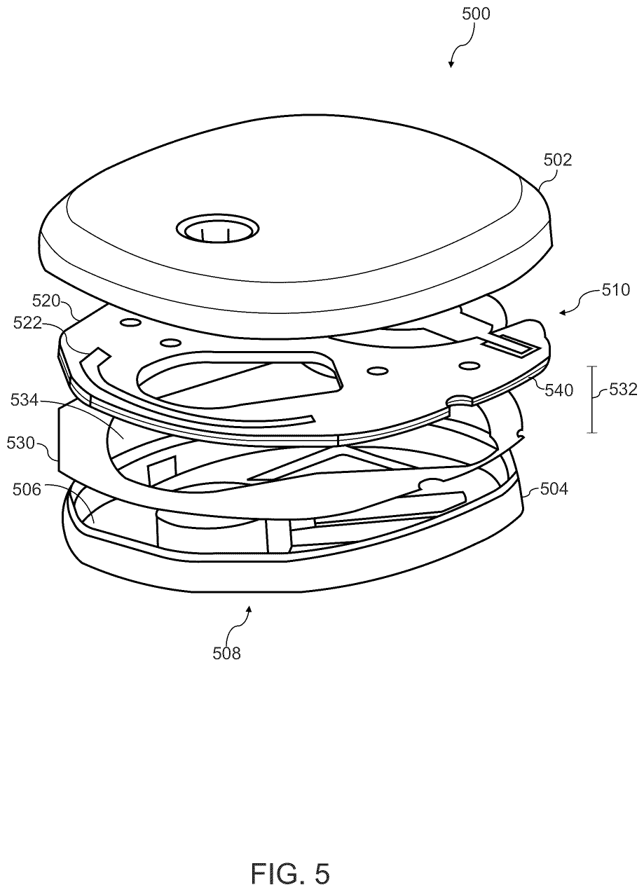

Referring now to FIG. 5, an exploded view of an example glucose monitoring device 500 is provided according to example embodiments of the present disclosure. As shown in FIG. 5, glucose monitoring device 500 includes a conductive structure 530 that is not connected to the circuit board

Glucose monitoring device 500 may further include a housing, such as a dielectric housing, having a first portion 502 and a second portion 504. In some embodiments, the first portion 502 and/or the second portion 504 of the housing may include a plastic material. Second portion 504 of the housing may be operable to be placed adjacent to a human body during operation of the glucose monitoring device 500 (e.g., glucose monitoring device). For instance, second portion 504 of the housing may have a first surface 506 and a second surface 508. First surface 506 (e.g., internal surface) may face away from a human body during operation of the glucose monitoring device 500, while second surface 508 may face toward a human body during operation of the glucose monitoring device 500. In some embodiments, an adhesive patch may be positioned on second surface 508, such that the adhesive patch is positioned between glucose monitoring device 500 and a human body (e.g., skin of a human body) during operation of glucose monitoring device 500.

Glucose monitoring device 500 may further include an antenna assembly 510. As shown in FIG. 5, antenna assembly 510 may include a circuit board 520 positioned between the first portion 502 and the second portion 504 of the housing. In some embodiments, a trace antenna 522 may be associated with circuit board 520. For instance, circuit board 520 may be a plastic circuit board (PCB) that includes a trace antenna 522.

Antenna assembly 510 may further include a conductive structure 530 that is spaced apart from the circuit board 520 and trace antenna 522. Specifically, conductive structure 530 may not be connected to circuit board 520. For example, conductive structure 530 may be formed on (e.g., mounted to) the second portion 504 of the housing such that the conductive structure 530 is not physically touching the circuit board 520. In some embodiments, the conductive structure 530 may be spaced apart from the circuit board 520 by a distance 532 of about 1 millimeter (mm) to about 2.5 mm. While FIG. 5 generally depicts an exploded view of glucose monitoring device 500, those of ordinary skill in the art will understand that distance 532 may depict the distance between the conductive structure 530 and the circuit board 520 during operation of the glucose monitoring device 500. Conductive structure 530 may also have an opening 534 to accommodate at least a portion of a sensor at least partially extending from the glucose monitoring device 500.

Glucose monitoring device 500 (e.g., antenna assembly 510) may be operable to communicate signals in the 2.4 GHz frequency band. In some examples, the glucose monitoring device 500 (e.g., antenna assembly 510) may be operable to communicate signals with a remote device (e.g., a smartphone or other device) via a short-range wireless communication protocol (e.g., Bluetooth communication protocol). In some examples, the glucose monitoring device 500 (e.g., antenna assembly 510) may be operable to communicate signals with a remote device (e.g., a smartphone or other device) via an 802.11 communication protocol. Other suitable communication protocols may be used without deviating from the scope of the present disclosure.

In some embodiments, conductive structure 530 may be a stamped metal conductive structure. For instance, conductive structure 530 may be a stamped metal antenna, such as stamped metal antenna 114 described with reference to FIG. 1. Conductive structure 530 may be positioned on second portion 504 of the housing such that the conductive structure 530 is spaced apart from the circuit board 520. Specifically, conductive structure 530 may not be operatively coupled to circuit board 520.

In alternative embodiments, conductive structure 530 may be a laser direct structuring (LDS) conductive structure. For instance, second portion 504 of the housing may act as a carrier (e.g., LDS carrier) for the conductive structure. A laser device may be used to etch one or more channels into an exterior surface of second portion 504. The conductive structure 530 may then be formed in the channels by, for example, placing second portion 504 in a metal bath such that the channels are filled with metal. As such, conductive structure 530 may be defined as an LDS conductive structure (e.g., LDS antenna element).

In some embodiments, antenna assembly 510 may further include a ground plane 540. Specifically, circuit board 520 may include ground plane 540 associated with an antenna (e.g., trace antenna) of the circuit board 520. For instance, ground plane 540 may be a layer of circuit board 520, such as a bottom layer as depicted in FIG. 5. The ground plane 540 may be associated with the trace antenna 522 of circuit board 520. In addition, the conductive structure 530 may be spaced apart (e.g., not connected to) the ground plane 540. For instance, the conductive structure 530 may be spaced apart from the ground plane 540 by distance 532.

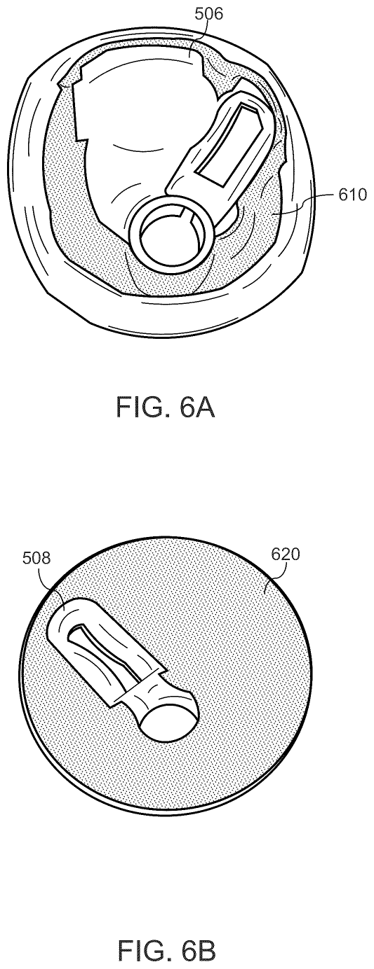

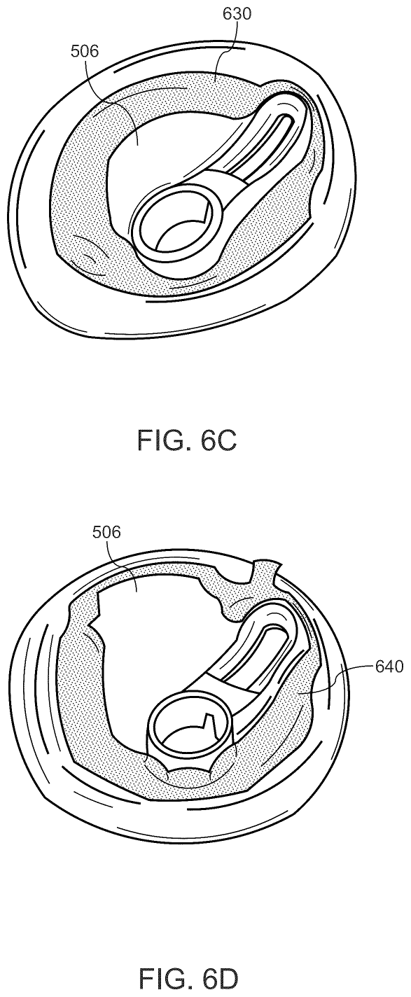

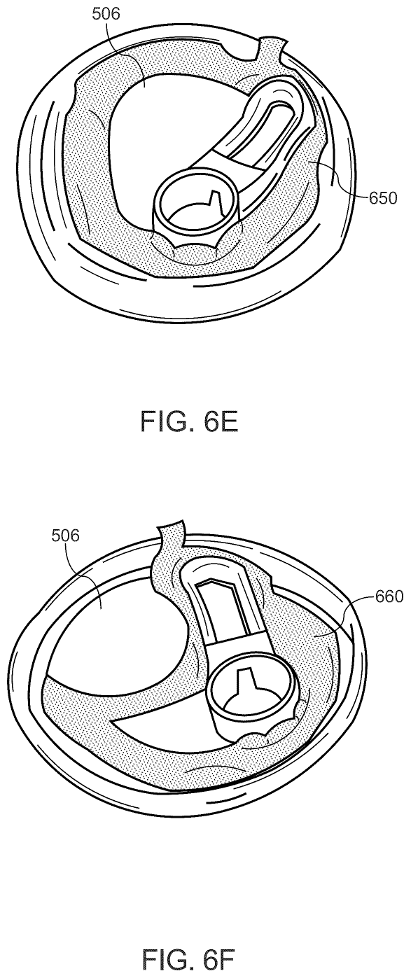

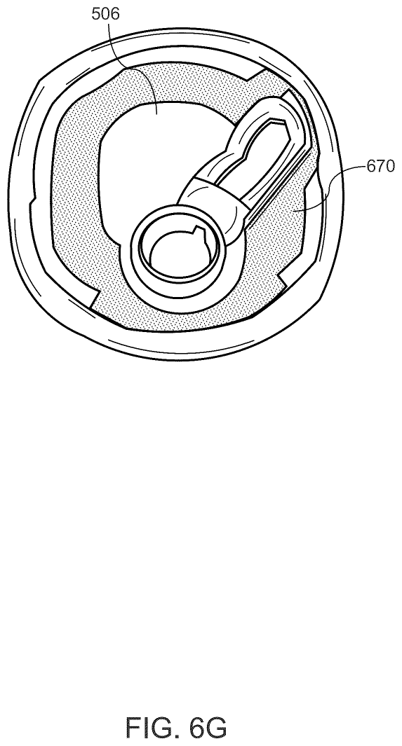

In some embodiments, conductive structure 530 may be positioned on a surface 506, 508 of the second portion 504 of the housing. While conductive structure 530 is depicted in FIG. 5 as being positioned between the circuit board 520 and the second portion 504 of the housing, those of ordinary skill in the art will understand that the conductive structure 530 may be positioned on the first surface 506 of the second portion 504 of the housing or the second surface 508 of the second portion 504 of the housing. For instance, FIG. 6A-6G provide perspective views of example conductive structures 610, 620, 630, 640, 650, 660, 670 positioned on a second portion of the housing, such as second portion 504 depicted in FIG. 5.

Referring now to FIG. 6A, an example conductive structure 610 of a glucose monitoring device according to example embodiments of the present disclosure is provided. As shown in FIG. 6A, conductive structure 610 may be positioned on a first surface 506 of the second portion of the housing. Conductive structure 610 may be a stamped metal conductive structure or an LDS conductive structure. For instance, the conductive structure 610 may be a stamped metal conductive structure at least partially positioned on first surface 506 of a second portion of the housing or an LDS conductive structure at least partially formed on the first surface 506 of a second portion of the housing.

The conductive structure 610 may be spaced apart from (e.g., not connected to) a circuit board of the glucose monitoring device or a ground plane associated with the circuit board, such as ground plane 540 (FIG. 5), of the circuit board.

Referring now to FIG. 6B, an example conductive structure 620 of a glucose monitoring device according to example embodiments of the present disclosure is provided. As shown in FIG. 6B, conductive structure 620 may be positioned on a second surface 508 of the second portion of the housing. As previously described with reference to FIG. 5, second surface 508 may face away from circuit board 520 (FIG. 5), such that conductive structure 620 may be positioned on an exterior surface of the housing. In some embodiments, an adhesive patch may be placed between the conductive structure 620 and a human body (e.g., skin of a human body) during operation of the glucose monitoring device.

Conductive structure 620 may be a stamped metal conductive structure or an LDS conductive structure. For instance, the conductive structure 620 may be a stamped metal conductive structure at least partially positioned on second surface 508 of a second portion of the housing or an LDS conductive structure at least partially formed on the second surface 508 of a second portion of the housing.

The conductive structure 620 may be spaced apart from (e.g., not connected to) a circuit board of the glucose monitoring device or a second conductive structure of the circuit board. For instance, a second portion 504 (FIG. 5) of the housing may be positioned between the conductive structure 620 and the circuit board 520 (FIG. 5). In some embodiments, conductive structure 620 may be positioned on (e.g., formed on) at least about 80% of the surface area of surface 508, such as at least about 90% of the surface area of surface 508, such as at least about 100% of the surface area of surface 508.

FIGS. 6C-6G provide various additional conductive structures 630, 640, 650, 660, 670 positioned at least partially on first surface 506 of a second portion of the housing of an example glucose monitoring device according to example embodiments of the present disclosure. As shown in FIGS. 6C-6G, conductive structures 630, 640, 650, 660, 670 may be stamped metal conductive structures or LDS conductive structures. For instance, the conductive structures 630, 640, 650, 660, 670 may be a stamped metal conductive structure at least partially positioned on first surface 506 of a second portion of the housing or an LDS conductive structure at least partially formed on the first surface 506 of a second portion of the housing.

Each conductive structure 630, 640, 650, 660, 670 may be spaced apart from (e.g., not connected to) a circuit board of the glucose monitoring device or a ground plane, such as ground plane 540 (FIG. 5), of the circuit board.

One example aspect of the present disclosure is directed to a glucose monitoring device. The glucose monitoring device includes a housing having a first portion and a second portion, the second portion operable to be placed adjacent to a human body during operation of the glucose monitoring device. The glucose monitoring device further includes a circuit board positioned between the first portion and the second portion of the housing, the circuit board having a trace antenna. The glucose monitoring device further includes a conductive structure spaced apart from the circuit board.

In some examples, the conductive structure is positioned between the second portion of the housing and the circuit board.

In some examples, the conductive structure is positioned between the second portion of the housing and the human body during operation of the glucose monitoring device.

In some examples, the conductive structure is a stamped metal conductive structure.

In some examples, the conductive structure is a laser direct structuring (LDS) conductive structure.

In some examples, the circuit board further comprises ground plane associated with the trace antenna.

In some examples, the conductive structure is not connected to the ground plane associated with the trace antenna.

In some examples, the conductive structure is spaced apart from the circuit board by a distance, the distance being in a range of about 1 millimeter (mm) to about 2.5 mm.

In some examples, the conductive structure has an opening to accommodate at least a portion of a sensor at least partially extending from the glucose monitoring device.

In some examples, the glucose monitoring device is operable to communicate signals over a 2.4 GHz frequency band.

Another example aspect of the present disclosure is directed to an antenna assembly for a glucose monitoring device. The antenna assembly includes a circuit board positioned between a first portion and a second portion of a housing, the second portion of the housing operable to be placed adjacent to a human body during operation of the glucose monitoring device. The antenna assembly further includes a trace antenna associated with the circuit board. The antenna assembly further includes a conductive structure positioned between the second portion of the housing and the circuit board. The conductive structure is spaced apart from the circuit board.

In some examples, the conductive structure is a stamped metal conductive structure.

In some examples, the conductive structure is a laser direct structuring (LDS) conductive structure.

In some examples, the conductive structure is spaced apart from the circuit board by a distance, the distance being in a range of about 1 millimeter (mm) to about 2.5 mm.

In some examples, the conductive structure has an opening to accommodate at least a portion of a sensor at least partially extending from the glucose monitoring device.

Another example aspect of the present disclosure is directed to a glucose monitoring device. The glucose monitoring device includes a plastic housing having a first portion and a second portion, the second portion operable to be placed adjacent to a human body during operation of the glucose monitoring device. The glucose monitoring device further includes a circuit board having a trace antenna positioned between the first portion and the second portion. The glucose monitoring device further includes a laser direct structuring (LDS) conductive structure formed on the second portion of the housing. The LDS conductive structure is spaced apart from the circuit board.

In some examples, the LDS conductive structure is at least partially formed on a first surface of the second portion of the housing, the first surface facing the circuit board.

In some examples, the LDS conductive structure is at least partially formed on a second surface of the second portion of the housing, the second surface of the second portion of the housing facing away from the circuit board.

In some examples, the LDS conductive structure is spaced apart from the circuit board by a distance, the distance being in a range of about 1 millimeter (mm) to about 2.5 mm.

In some examples, the LDS conductive structure has an opening to accommodate at least a portion of a sensor at least partially extending from the glucose monitoring device.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A glucose monitoring device, comprising:
a housing having a first portion and a second portion, the second portion operable to be placed adjacent to a human body during operation of the glucose monitoring device;
a circuit board positioned between the first portion and the second portion of the housing, the circuit board having a trace antenna; and
a conductive structure spaced apart from the circuit board.

2. The glucose monitoring device of claim 1, wherein the conductive structure is positioned between the second portion of the housing and the circuit board.

3. The glucose monitoring device of claim 1, wherein the conductive structure is positioned between the second portion of the housing and the human body during operation of the glucose monitoring device.

4. The glucose monitoring device of claim 1, wherein the conductive structure is a stamped metal conductive structure.

5. The glucose monitoring device of claim 1, wherein the conductive structure is a laser direct structuring (LDS) conductive structure.

6. The glucose monitoring device of claim 1, wherein the circuit board further comprises ground plane associated with the trace antenna.

7. The glucose monitoring device of claim 6, wherein the conductive structure is not connected to the ground plane associated with the trace antenna.

8. The glucose monitoring device of claim 1, wherein the conductive structure is spaced apart from the circuit board by a distance, the distance being in a range of about 1 millimeter (mm) to about 2.5 mm.

9. The glucose monitoring device of claim 1, wherein the conductive structure has an opening to accommodate at least a portion of a sensor at least partially extending from the glucose monitoring device.

10. The glucose monitoring device of claim 1, wherein the glucose monitoring device is operable to communicate signals over a 2.4 GHz frequency band.

11. An antenna assembly for a glucose monitoring device, comprising:
a circuit board positioned between a first portion and a second portion of a housing, the second portion of the housing operable to be placed adjacent to a human body during operation of the glucose monitoring device;
a trace antenna associated with the circuit board; and
a conductive structure positioned between the second portion of the housing and the circuit board,
wherein the conductive structure is spaced apart from the circuit board.

12. The antenna assembly of claim 11, wherein the conductive structure is a stamped metal conductive structure.

13. The antenna assembly of claim 11, wherein the conductive structure is a laser direct structuring (LDS) conductive structure.

14. The antenna assembly of claim 11, wherein the conductive structure is spaced apart from the circuit board by a distance, the distance being in a range of about 1 millimeter (mm) to about 2.5 mm.

15. The antenna assembly of claim 11, wherein the conductive structure has an opening to accommodate at least a portion of a sensor at least partially extending from the glucose monitoring device.

16. A glucose monitoring device, comprising:
a plastic housing having a first portion and a second portion, the second portion operable to be placed adjacent to a human body during operation of the glucose monitoring device;
a circuit board having a trace antenna positioned between the first portion and the second portion; and
a laser direct structuring (LDS) conductive structure formed on the second portion of the housing,
wherein the LDS conductive structure is spaced apart from the circuit board.

17. The glucose monitoring device of claim 16, wherein the LDS conductive structure is at least partially formed on a first surface of the second portion of the housing, the first surface facing the circuit board.

18. The glucose monitoring device of claim 16, wherein the LDS conductive structure is at least partially formed on a second surface of the second portion of the housing, the second surface of the second portion of the housing facing away from the circuit board.

19. The glucose monitoring device of claim 16, wherein the LDS conductive structure is spaced apart from the circuit board by a distance, the distance being in a range of about 1 millimeter (mm) to about 2.5 mm.

20. The glucose monitoring device of claim 16, wherein the LDS conductive structure has an opening to accommodate at least a portion of a sensor at least partially extending from the glucose monitoring device.
